(19) United States
(12) Patent Application Publication
(10) Pub. No.: US 2002/0123090 A1
(43) Pub. Date: Sep. 5, 2002

(54) Ultrapure vegetable protein materials

(75) Inventors: Theodore M. Wong, Manchester, MO (US); David A. Singer, St. Louis, MO (US); Santa H. Lin, Millfield Court Town ?amp; Country, MO (US); Terry T. Lin, Town ?amp; Country, MO (US)

Correspondence Address: Richard B. Taylor, P. O. Box 88940, St. Louis, MO, 63188, US

(73) Assignee: Protein Technologies International INc., St. Louis, MO (US)

(21) Appl. No.: 09/912,471

(22) Filed: Jul. 24, 2001

Related U.S. Application Data
(63) Continuation-in-part of application No. 08/996,976, filed on Dec. 23, 1997, now abandoned.

(51) Int. Cl. C12P 21/06

(52) U.S. Cl. 435/68.1

(57) ABSTRACT

A method of reducing the ribonucleic acid content in a vegetable protein material is provided. A vegetable material containing protein and ribonucleic acids is treated with an enzyme preparation containing an acid phosphatase to degrade the ribonucleic acids in the vegetable protein material.

### FIELD OF THE INVENTION

This invention relates to methods for producing purified vegetable protein materials, and more particularly, to methods for producing ultrapure vegetable protein isolates and concentrates.

### BACKGROUND OF THE INVENTION

Many food and beverage products include protein supplements derived from vegetable materials such as soybeans, beans, peas, other legumes, and oilseeds such as rapeseed. Vegetable protein materials, particularly soy, are used to fortify infant formulas. The purpose of the vegetable protein supplement in an infant formula is to increase the nutritional value of the formula, and to provide a protein content approximate to the protein content of human milk.

Commercially available protein concentrates and isolates, however, contain some impurities which are undesirable in products such as infant formulas. Specific impurities which are undesirable in vegetable protein isolates and concentrates include phytic acid, phytates, ribonucleic acids, ash, and minerals bound to phytic acid, phytates, or ribonucleic acids which are unavailable for human assimilation such as phosphorus, calcium, chloride, iron, zinc, and copper. It is desirable to provide methods for reducing the levels of these impurities in vegetable protein isolates and concentrates, particularly for use in products such as infant formulas.

Reducing the level of phytic acid, also known as inositol hexaphosphoric acid, and phytates, which are the salts of phytic acid, in vegetable protein materials has been of interest since phytic acid and phytates tend to form complexes with proteins and multivalent metal cations, reducing the nutritional value of the vegetable protein material. Significant efforts have been made to reduce the concentration of phytic acid and phytates in vegetable protein materials. For example, U.S. Pat. No. 5,248,765 to Mazer et al. provides a method of separating phytate and manganese from protein and dietary fiber by treating an aqueous slurry of the phytate-containing material with alumina at low pH. The alumina, together with phytate attached to the alumina, is then separated from the protein and fiber material. U.S. Pat. No. 2,732,395 to Bolley et al., U.S. Pat. No. 4,072,670 to Goodnight et al., U.S. Pat. No. 4,088,795 to Goodnight et al., U.S. Pat. No. 4,091,120 to Goodnight et al., and U.K. Patent No. 1,574,110 to deRham all teach various methods of removing phytic acid and phytates from protein materials by various precipitation and differential solubility separation techniques.

Other methods of reducing phytic acid or phytate concentrations in vegetable protein materials utilize enzymes to degrade phytic acid or phytates. European Patent Application No. 0 380 343 A2 provides a method of preparing phytate-free or low phytate soy protein isolates and concentrates in which a phytic acid and a phytate degrading enzyme (hereinafer a “phytase”) is added to a soy protein isolate or concentrate at a temperature of 20° C. to 60° C. and at a pH of 2-6 to degrade phytic acid and phytates in the protein material. U.S. Pat. No. 4,642,236 to Friend et al, U.S Pat. No. 3,733,207 to McCabe, and Japanese Kokai Patent Application No. Hei 8[1996]-214787 all provide processes in which phytases are used to degrade phytic acid and phytates in soy protein. Phytase enzyme preparations are particularly useful for purifying vegetable protein materials since they are inexpensive and readily commercially available.

Phytase enzymes are phosphoric monoester hydrolases (I.U.B. 3.1.3) and are usually derived from microbial or fungal sources such as the Aspergillus and Rhizopus species. Commonly used phytase enzyme compositions typically include the enzyme 3-phytase (myo-inositol-hexakisphosphate 3-phosphohydrolase (I.U.B. 3.1.3.8)) as a primary phytase enzyme. Some, but not all, phytase enzyme compositions include sufficient concentrations of the enzyme acid phosphatase (orthophosphoric monoester phosphohydrolase (I.U.B. 3.1.3.2)) to effect degradation of phytic acid and phytates.

Phytase enzyme compositions are not recognized to reduce the levels of ribonucleic acid materials and associated minerals in vegetable protein materials since the most common phytases, especially 3-phytase, do not degrade the ribonucleic acid structure. Ribonuclease enzyme compositions are known to cleave and degrade ribonucleic acids, and can be used to reduce levels of ribonucleic acids in vegetable proteins, however, such enzyme compositions are quite expensive and are impractical for use on a scale necessary for commercial production of purified vegetable protein materials.

It is desirable to reduce the levels of ribonucleic acid materials and associated minerals in vegetable proteins at costs which make the methods practical for use on a commercial scale.

### SUMMARY OF THE INVENTION

In one aspect, the invention is a method for reducing the concentrations of ribonucleic acids and minerals bound to ribonucleic acids from a vegetable protein material. A vegetable protein material is provided and is slurried in an aqueous solution. The slurry is treated with an enzyme preparation containing an acid phosphatase at a pH and a temperature and for a time effective to substantially reduce the ribonucleic acid concentrations in the vegetable protein material. The treated slurry is then washed to provide a vegetable protein material having a reduced concentration of ribonucleic acids.

In a preferred embodiment of the invention, the mineral content of the vegetable protein material are reduced by treatment of the vegetable protein material slurry with the enzyme preparation containing an acid phosphatase.

In another preferred embodiment of the invention, the vegetable protein material is a soy protein, the pH at which the slurry is treated with the enzyme preparation is from about 3 to about 6, the temperature at which the slurry is treated with the enzyme preparation is from about 20° C. to about 70° C., and the time period over which the slurry is treated with the enzyme preparation is from about 30 minutes to about 4 hours. The treated slurry is washed after being treated with the enzyme preparation.

In yet another preferred embodiment, the slurry is heat treated after being enzymatically treated and washed, and the heat treated slurry is dried.

In another aspect, the invention is a method for reducing the concentrations of phytic acid, phytates, ribonucleic acids, and minerals bound to phytic acid, phytates, and ribonucleic acids from a vegetable protein material. A vegetable protein material is provided and is slurried in an aqueous solution. The slurry is treated with an enzyme preparation containing an acid phosphatase and a phytase at a pH and a temperature and for a time effective to substantially reduce the phytic acid, phytate, and ribonucleic acid concentrations in the vegetable protein material.

### DESCRIPTION OF THE PREFERRED EMBODIMENTS

The present invention resides in the discovery that acid phosphatase enzymes unexpectedly cleave ribonucleic acids, and therefore can be used to degrade and reduce the concentration of ribonucleic acid materials in vegetable protein materials on a commercial scale, as well as remove minerals and ash bound by the ribonucleic acid materials. Although certain commercially available phytase enzyme preparations include acid phosphatases, it has not been previously recognized that acid phosphatases are useful for degrading ribonucleic acids, and that the concentration of ribonucleic acids in vegetable protein materials can be reduced by treatment with an acid phosphatase. Acid phosphatase is capable of degrading phytic acid and phytates as well as ribonucleic acids, therefore, an acid phosphatase can be used to degrade and reduce the concentrations of phytic acid and phytates as well as ribonucleic acids, or can be used in combination with other phytases.

The starting material for the process of the present invention is a vegetable protein concentrate or a vegetable protein isolate. As used herein, and according to conventional definition, a vegetable protein concentrate is a vegetable protein material containing 65%-90% protein on a dry basis, and a vegetable protein isolate is a vegetable protein material containing at least 90% protein on a dry basis. Vegetable protein concentrates and isolates are readily commercially available. For example, soy protein isolates which may be used in the process of the present invention are available from Protein Technologies International, Inc., St. Louis, Mo., and are sold under the trade names SUPRO® 500E and SUPRO® 620.

Vegetable protein concentrates and vegetable protein isolates may be prepared according to conventional methods. Vegetable protein concentrates are commonly prepared by (i) leaching a vegetable protein material with an aqueous solution having a pH at about the pH of the isoelectric point of the protein; (ii) extracting a vegetable protein material with an aqueous alcohol; or (iii) denaturing a vegetable protein material with moist heat, followed by extraction of the denatured vegetable protein material with water.

In a preferred embodiment, a soy protein concentrate is prepared for use in the method of the present invention. Commercially available defatted soy flakes (dehulled and defatted soybeans) are washed with an aqueous solution having a pH at about the isoelectric point of soy protein, preferably at a pH of about 4 to about 5, and most preferably at a pH of about 4.4 to about 4.6. The aqueous acidic solution leaches water soluble carbohydrates, minerals, phenolics, and other non-proteinaceous materials away from the soy protein, which is insoluble in the aqueous solution at its isoelectric point, leaving the soy protein concentrate.

Vegetable protein isolates are formed by extracting a vegetable protein material with an aqueous alkaline solution to solubilize protein material. The solubilized protein material extract is then separated from insoluble vegetable matter such as cellulose and other vegetable fibers. The pH of the protein extract is then adjusted to about the isoelectric point of the protein to precipitate the protein. The precipitated protein is separated from the solution by filtration or centrifugation to separate the protein material from water soluble carbohydrates, minerals, phenolics, and other non-proteinaceous materials which remain in the solution. The separated protein is then washed with water to form the protein isolate.

In a most preferred embodiment, a soy protein isolate is prepared for use in the method of the present invention. Commercially available defatted soy flakes are utilized as the starting material. Preferably the soy flakes have been treated with a sulfite such as sodium sulfite for improved flow characteristics and improved microbial control. The soy flakes are extracted with an aqueous alkaline solution, preferably an aqueous sodium hydroxide solution, having a pH from about 8 to about 11. Preferably the weight ratio of the extractant to the soy flake material is from about 5:1 to about 16:1. The extract is separated from the insoluble materials such as soy fiber and cellulose by filtration or by centrifugation and decantation of the supernatant extract from the insoluble materials. The pH of the separated extract is adjusted to about the isoelectric point of soy protein, preferably from about pH 4 to about pH 5, most preferably from about pH 4.4 to about pH 4.6, with a suitable acid, preferably hydrochloric acid, sulfuric acid, nitric acid, or acetic acid, to precipitate a soy protein material. The precipitated protein material is separated from the extract, preferably by centrifugation or filtration. The separated protein material is washed with water, preferably at a weight ratio of water to protein material of about 5:1 to about 12:1 to produce the soy protein isolate.

An aqueous slurry of the vegetable protein concentrate or vegetable protein isolate (hereinafter, generally, the “protein material”) is formed by mixing the protein material with water to form a slurry. Preferably the slurry should contain from about 2% to about 30% of the protein material by weight, and more preferably should contain from about 5% to about 20% of the protein material by weight, and most preferably should contain from about 10% to about 18% of the protein material by weight.

The slurry is then treated with an enzyme preparation containing an acid phosphatase (orthophosphoric monoester phosphohydrolase (I.U.B. 3.1.3.2)) at an acid phosphatase concentration, temperature, a pH, and for a time effective to substantially reduce the concentration of ribonucleic acids in the protein material. The enzyme preparation containing an acid phosphatase is derived from a microbial or fungal source such as the Aspergillus and Rhizopus species. A preferred source of the acid phosphatase useful in the method of the present invention is the Aspergillus niger fungus. Phytase enzyme preparations derived from Aspergillus niger and which contain acid phosphatase are commercially available.

The enzyme preparation is added to the slurry in sufficient amount to provide an acid phosphatase concentration effective to degrade and substantially reduce the concentration of ribonucleic acids present in the protein material. Preferably at least a majority of the ribonucleic acids present in the initial vegetable protein material are degraded by the acid phosphatase enzyme, where the term a majority is defined to be 50% or greater. More preferably, the acid phosphatase degrades at least 60% of the ribonucleic acids in the vegetable protein material, even more preferably at least 70% of the ribonucleic acids in the protein material, and even more preferably at least 80% of the ribonucleic acids in the protein material, and most preferably the acid phosphatase degrades substantially all of the ribonucleic acids in the protein material.

In order to effectively degrade and reduce the concentration of the ribonucleic acids in the protein material, the enzyme preparation should include a sufficient amount of acid phosphatase, or a combination of acid phosphatase and another phytase such as 3-phytase(myo-inositol-hexakisphosphate 3-phosphohydrolase (I.U.B. 3.1.3.8)), to degrade and substantially reduce the concentration of the ribonucleic acids. Preferably the enzyme preparation is added so that the acid phosphatase is present in the slurry from about 0.1% to about 10% of the protein material by dry weight, more preferably from about 0.3% to about 5% of the protein material by dry weight, and most preferably from about 0.5% to about 3% of the protein material by dry weight.

In the most preferred embodiment of the invention the enzyme preparation degrades and reduces the concentration of phytic acid and phytates as well as ribonucleic acids. Preferably the enzyme preparation degrades at least a majority of the phytic acid and phytates, where a majority is defined as 50%, more preferably at least 75% of the phytic acid and phytates are degraded, even more preferably at least 85% of the phytic acid and phytates are degraded, and most preferably substantially all of the phytic acid and phytates are degraded by the enzyme preparation.

In order to effectively degrade and reduce the concentration of the ribonucleic acids, phytic acid, and phytates in the protein material, the enzyme preparation should include a sufficient amount of acid phosphatase, or a combination of acid phosphatase and another phytase such as 3-phytase(myo-inositol-hexakisphosphate 3-phosphohydrolase (I.U.B. 3.1.3.8)) to degrade the ribonucleic acids, phytic acid, and phytates. In a most preferred embodiment, the enzyme preparation is added so that the acid phosphatase and 3-phytase are present in the slurry from about 0.1% to about 10% of the protein material by dry weight, more preferably from about 0.3% to about 5% of the protein material by dry weight, and most preferably from about 0.5% to about 3% of the protein material by dry weight.

The activity of the enzyme preparation should be effective to degrade and substantially reduce the concentration of ribonucleic acids, the phytic acid concentration, and the concentration of phytates. The enzyme preparation preferably has an activity from about 400 to about 1400 kilo phytase units per kilogram of protein solids (KPU/kg protein solid), more preferably has an activity of about 600 to about 1200 KPU/kg protein solid, and most preferably has an activity of about 1000 KPU/kg protein solid. A kilo phytase unit equals 1000 phytase units, where a phytase unit equals the quantity of enzyme which liberates one nanomole of inorganic phosphates from sodium phytate in one minute under standard conditions (40° C., pH 5.5, and 15 minutes incubation). The activity of the enzyme preparation includes acid phosphatase activity and the activity of any other phytase enzyme included in the enzyme preparation.

The pH of the slurry treated with the enzyme preparation should be a pH at which the enzyme preparation is effective to degrade ribonucleic acids, and preferably, a pH at which the enzyme preparation also degrades phytic acid and phytates. It has been discovered that acid phosphatase enzymes very effectively degrade ribonucleic acids in vegetable protein materials at a pH of about 4.5, and it is known in the art that phytase enzymes very effectively degrade phytic acid and phytates at a pH of about 5.3. In a preferred embodiment, the pH of the slurry treated with the enzyme preparation is from about 3 to about 6, more preferably from about 3.5 to about 5.5, and even more preferably from about 4 to about 5, and most preferably from about 4.4 to about 4.6. The pH of the slurry may be adjusted with a suitable acidic reagent, such as hydrochloric acid, sulfuric acid, nitric acid, or acetic acid, or a suitable basic reagent, such as sodium hydroxide, calcium hydroxide or ammonium hydroxide, as necessary to obtain the desired pH.

The temperature of the slurry treated with the enzyme preparation should be a temperature at which the enzymes in the enzyme preparation are effective to degrade ribonucleic acids, and preferably also degrade phytic acid and phytates. Preferably the temperature of the slurry should be high enough to maximize the enzymatic degradation of the ribonucleic acids, phytic acid, and phytates, but not high enough to inactivate the enzyme(s) or to degrade the protein material in the slurry. In a preferred embodiment, the temperature at which the slurry is treated with the enzyme preparation containing acid phosphatase is from about 20° C. to about 70° C., more preferably from about 30° C. to about 60° C., and most preferably from about 40° C. to about 55° C.

The time period which the slurry is treated with the enzyme preparation should be sufficient to enable the enzyme(s) to effectively degrade and reduce the concentration of ribonucleic acids, and preferably also degrade and reduce the concentrations of the phytic acid and phytates in the vegetable protein material. Preferably the slurry is treated with the enzyme preparation at an effective pH and temperature from about 30 minutes to about 4 hours, more preferably from about 45 minutes to about 3 hours, and most preferably from about 1 hour to about 2 hours.

Following treatment of the vegetable protein material slurry with the enzyme preparation, the vegetable protein material is washed to remove the degraded materials, ash, and minerals. Preferably the vegetable protein material is washed by diluting the vegetable protein material slurry with water and centrifuging the diluted slurry. More preferably the vegetable protein material is washed twice, for example, by diluting the vegetable protein material slurry with water, centrifuging the diluted slurry in a disc centrifuge, and then centrifuging the slurry in a bowl centrifuge.

Most preferably, the pH of the slurry in the wash step is about the isoelectric point of the vegetable protein material after degradation of the ribonucleic acids, phytic acid, and phytates to minimize loss of protein material in the wash. Degradation of the ribonucleic acids, phytic acid, and phytates may cause the isoelectric point of the protein material to shift. For example, soy protein including ribonucleic acids, phytic acid, and phytates has an isoelectric point of about pH 4.5, but has an isoelectric point of about pH 5.1 after enzymatic degradation of these materials. The pH of the slurry may be adjusted to about the isoelectric point of the protein material, if necessary, with a suitable acidic or basic reagent prior to washing the protein material.

The wash should be conducted with sufficient amounts of wash water, preferably pH adjusted to about the isoelectric point of the protein, to remove the degraded ribonucleic acids, and preferably, the degraded phytic acid and phytates, from the vegetable protein material. In a preferred embodiment, at least a majority of the degraded ribonucleic acids, phytic acid, and phytates present in the initial vegetable protein material are removed by the process of the present invention, where the term “majority” is defined as 50% or greater. More preferably, the process of the present invention is effective to remove at least 60% of the degraded ribonucleic acids, phytic acid, and phytates present in the vegetable protein material, even more preferably at least 70% of the degraded ribonucleic acids, phytic acid, and phytates present in the vegetable protein material, and even more preferably at least 80% of the degraded ribonucleic acids, phytic acid, and phytates present in the vegetable protein material, and most preferably substantially all of the degraded ribonucleic acids, phytic acid, and phytates present in the vegetable protein material are removed.

After washing, a purified vegetable protein material may be recovered from the slurry by drying the protein material. In a preferred embodiment, the purified vegetable protein material is recovered by spray drying the protein material in accordance with conventional spray drying techniques.

The vegetable protein material having reduced levels of ribonucleic acids, and preferably, reduced levels of phytic acid and phytates, may be processed further, if desired, to provide a purified protein material with modified functional characteristics. The slurry of purified protein material may be heat treated to denature the protein and to sterilize the protein material. Preferably the slurry is heat treated by jet cooking in accordance with conventional jet cooking techniques, and is flash cooled by ejection from a jet cooker into a vacuumized chamber. Most preferably, the slurry of purified protein material is heat treated under pressure at a temperature of about 140° C. to about 160° C. for a period of about 1 to 15 seconds. In a most preferred embodiment, the pH of the protein slurry is neutralized to a pH of about 6 to about 8 with a suitable basic reagent, preferably an aqueous sodium hydroxide/potassium hydroxide solution, prior to heat treating the slurry to aid in processing the heated treated protein material.

The purified protein material, either heat treated or untreated, may also be subjected to enzymatic hydrolysis to reduce the viscosity of the protein material. Enzymatic hydrolysis is particularly desirable after heat treatment of the protein material since the denatured protein material is more viscous than similar protein material which has not been subjected to a heat treatment. A slurry of the purified protein material may be treated with a conventional, commercially available protease enzyme at a pH, a temperature, an enzyme concentration and activity, and for a time effective to hydrolyze the protein material.

The pH at which the enzymatic hydrolysis is effected is dependent on the particular protease enzyme used. A protease enzyme should be selected to effect the hydrolysis which has a known pH range at which the enzyme is effective to hydrolyze protein, and the hydrolysis of the purified protein material should be conducted within the known effective pH range of the enzyme. In a preferred embodiment, the protease enzyme Bromelain is utilized at a pH of from about 4 to about 9.

The concentration and activity of the protease should be sufficient to effect the desired degree of hydrolysis of the protein. Preferably the protease is added to a slurry of the purified protein material so that the protease is present in about 0. 1% to about 10% of the protein material by dry weight, and more preferably in about 0.5% to about 5% of the protein material by dry weight. Further, preferably, the protease should have an activity of from about 1000 to 4000 Tyrosine Units per gram (“TU/g”), and more preferably should have an activity of about 2000 to about 3000 TU/g, where 1 TU/g equals the enzyme activity which liberates one micromole of tyrosine per minute at 30° C. after 15 minutes of incubation at the protease's optimum pH for effecting hydrolysis of a protein material.

The temperature of the slurry treated with the protease should be a temperature at which the protease is effective to hydrolyze the purified protein material. Preferably the temperature of the slurry should be high enough to maximize the enzymatic hydrolysis of the protein material, but not high enough to inactivate the enzyme. In a preferred embodiment, the temperature at which the slurry is treated with the protease is from about 15° C. to about 75° C., more preferably from about 30° C. to about 65° C., and most preferably from about 40° C. to about 55° C.

The time period which the slurry is treated with the protease should be sufficient to enable the enzyme to hydrolyze the protein material to the desired degree of hydrolysis. Preferably the slurry is treated with the protease at an effective pH and temperature from about 15 minutes to about 2 hours, more preferably from about 30 minutes to about 1.5 hours, and most preferably from about 45 minutes to about 1 hour. After the enzyme hydrolysis is complete, the reaction is quenched by heating the slurry to a temperature above the inactivation temperature of the protease, for example, by heating the slurry to a temperature above 75° C.

The hydrolyzed purified vegetable protein material may be heat treated, if desired to sterilize the protein material and to denature the hydrolyzed protein material, if the protein material has not previously been heat treated. Preferably the slurry is heat treated by jet cooking in accordance with conventional jet cooking techniques, and is flash cooled by ejection from a jet cooker into a vacuumized chamber. Most preferably, the slurry of hydrolyzed purified protein material is heat treated under pressure at a temperature of about 140° C. to about 160° C. for a period of about 1 to 15 seconds.

After enzymatic hydrolysis, and, optionally, heat treatment, the hydrolyzed purified protein material may be recovered from the slurry by drying the protein material. In a preferred embodiment, the hydrolyzed purified vegetable protein material is recovered by spray drying the protein material in accordance with conventional spray drying techniques.

The following examples provide illustrations of the methods of the present invention, but are not to be interpreted as limiting the invention to the exemplified methods.

### EXAMPLE 1

A purified vegetable protein isolate is formed in accordance with the process of the present invention. Two hundred forty-three pounds of a soy protein isolate is added to two thousand nine hundred and fifty-nine pounds of water to form a soy protein isolate slurry containing 7.6% solids. The pH of the slurry is adjusted to 4.5 with hydrochloric acid, and the temperature of the slurry is raised to 50° C. An enzyme preparation containing an acid phosphatase and a phytase and having an activity of 1000 KPU/kg of curd solids is added to the slurry. The slurry is treated with the enzyme preparation for two hours, after which the pH of the slurry is adjusted to 5.1 with a caustic blend of potassium hydroxide and sodium hydroxide. The slurry is then diluted with water to a concentration of 4.2% solids, and is washed in a bowl centrifuge. Two hundred and seventy-five pounds of the washed slurry are neutralized with a caustic blend of potassium hydroxide and sodium hydroxide. The neutralized material is heat treated by jet cooking at 150° C. and flash cooled to 53° C. by ejection into a vacuumized chamber having a pressure of about 26 torr. The heat treated slurry is then spray dried to recover 15.5 pounds of purified soy protein isolate.

### EXAMPLE 2

A hydrolyzed purified vegetable protein isolate is formed in accordance with the process of the present invention. One thousand fifteen pounds of a purified soy protein isolate slurry containing 15.5% solids (approximately 157 pounds of purified soy protein material) is adjusted to pH 7.4 with 1400 milliliters of a sodium hydroxide/potassium hydroxide blend. The slurry is jet cooked to a temperature of 150° C. for 9 seconds and is flash cooled by ejection into a vacuumized chamber. Seven hundred twenty-five pounds of the slurry is treated with the protease enzyme Bromelain, the enzyme having an activity of 2500 TU/g and being added to the slurry to a concentration of 0.29% of the protein material in the slurry by dry weight. The temperature of the enzyme treated slurry is maintained at about 50° C. for the duration of the enzymatic treatment, which is 40 minutes. After the enzyme treatment the slurry is cooled to 16° C. and an additional 190 milliliters of the sodium hydroxide/potassium hydroxide blend is added to the slurry. The slurry is then jet cooked at 150° C. for 9 seconds and is flash cooled by ejection into a vacuumized chamber. The slurry is then spray dried to recover 75 pounds of a hydrolyzed purified soy protein isolate.

### EXAMPLE 3

The effect that enzyme activity has on ribonucleic acid concentration and phytic acid concentration in a soy protein isolate is examined, where the soy protein isolate is purified in a process performed in accordance with the present invention. A soy protein isolate slurry is formed by combining soy protein isolate and water adjusted to pH 4.5 with hydrochloric acid, where the total solids in the slurry are present in about 8.5% of the slurry by weight. The slurry is heated to a temperature of 50° C.

Two samples of the slurry are prepared from the protein isolate slurry for enzymatic degradation of ribonucleic acids and phytic acid, the first sample weighing 1530 lbs. and containing 8.66% total solids by weight, and the second sample weighing 1510 lbs. and containing 8.66% total solids by weight. Enzyme preparations containing an acid phosphatase and a phytase enzyme are added to each slurry sample. An enzyme preparation having an activity of 800 KPU/kg of curd solids is added to the first sample. An enzyme preparation having an activity of 1400 KPU/kg of curd solids is added to the second sample. The samples are reacted with the enzyme preparations for 1 hour. Following the enzyme treatment of the samples, the samples are thoroughly washed and the enzymes are thermally deactivated by jet cooking at a temperature of 150° C. The samples are flash cooled to 50° C. by ejection into a vacuumized chamber. The cooled samples are then spray dried.

A control sample having a total solids content of 7.6% is provided from the initial protein slurry for comparison purposes. The control sample is heated to 50° C. for 1 hour, and then is thoroughly washed. The washed control sample is jet cooked at 150° C. and then is flash cooled in a vacuumized chamber to 52° C. The control sample is then spray dried.

The samples are analyzed to determine the ribonucleic acid content and the phytic acid content of the samples. The results of the analysis are shown in Table 1 below.

TABLE 1

| | Phytic | Ribonucleic acid | % reduction of |
|---|---|---|---|
| KPU/kg curd solid | acid (%) | (mg/kg) | ribonucleic acid |
| 0 (control) | 1.4 | 9143 | — |
| 800 | 0.43 | 1784 | 80.5 |
| 1400 | 0.18 | 1769 | 80.7 |

The results clearly show that the enzyme preparations containing an acid phosphatase and a phytase and having an activity of 800 and 1400 KPU/kg of curd solids are effective to substantially reduce the ribonucleic acid content of a soy protein isolate. The results also show that the enzyme preparations are quite effective in reducing the phytic acid content of the protein isolate.

### EXAMPLE 4

The effect of pH on the enzymatic degradation of ribonucleic acids and phytic acid by an enzyme preparation containing an acid phosphatase and a phytase enzyme in a soy protein isolate is examined, where the soy protein isolate is purified in accordance with the present invention. A slurry is formed of soy protein isolate by mixing sufficient soy protein isolate with water adjusted to pH 4.5 by hydrochloric acid to form a slurry containing about 8% of the soy protein isolate by weight. The slurry is heated to a temperature of 50° C.

Two samples of the slurry containing 8% total solids by weight are prepared from the protein isolate slurry for enzymatic degradation of the ribonucleic acids and phytic acid. The first sample is adjusted to a pH of 5.1 with a potassium hydroxide/sodium hydroxide blend. The second sample is left at a pH of 4.5. These samples are then treated for two hours with an enzyme preparation containing an acid phosphatase enzyme and a phytase enzyme and having an activity of 1400 KPU/kg of curd solids. Following the enzyme treatment of the samples, the samples are thoroughly washed and the enzymes are thermally deactivated by jet cooking at a temperature of 150° C. The samples are flash cooled to 50° C. by ejection into a vacuumized chamber. The cooled samples are then spray dried.

A control sample having a total solids content of 7.6% is provided from the initial protein slurry for comparison purposes. The control sample is heated to 50° C. for 1 hour, and then is thoroughly washed. The washed control sample is jet cooked at 150° C. and then is flash cooled in a vacuumized chamber to 52° C. The control sample is then spray dried.

The samples are analyzed to determine the ribonucleic acid content and the phytic acid content of the samples. The results of the analysis are shown in Table 2 below.

TABLE 2

| | Phytic | Ribonucleic acid | % reduction of |
|---|---|---|---|
| pH | acid (%) | (mg/kg) | ribonucleic acid |
| 4.5 (control sample) | 1.4 | 9143 | — |
| 5.1 (sample 1) | 0.08 | 3180 | 65.3 |
| 4.5 (sample 2) | <0.07 | 1386 | 84.9 |

The results show that an enzyme preparation containing an acid phosphatase and a phytase are effective to substantially reduce both the phytic acid and ribonucleic acid content in a soy protein isolate at pH 4.5 and at pH 5.1. The reduction of the ribonucleic acid content in the protein isolate is particularly effective at pH 4.5.

### EXAMPLE 5

The effect of the time of enzymatic treatment on the enzymatic degradation of ribonucleic acids and phytic acid in soy protein isolate by an enzyme preparation containing an acid phosphatase and a phytase enzyme is examined, where the soy protein isolate is purified in accordance with the present invention. A slurry is formed of soy protein isolate by mixing sufficient soy protein isolate with water adjusted to pH 4.6 by hydrochloric acid to form a slurry containing about 8% of the soy protein isolate by weight. The slurry is heated to a temperature of 50° C.

Two samples of the slurry are prepared for enzymatic degradation of the ribonucleic acids and phytic acid. The first slurry sample weighs 3202 lbs. and contains 7.6% total solids by weight. The second sample weighs 1530 lbs. and contains 8.6% total solids by weight. An enzyme preparation containing an acid phosphatase and a phytase and having an activity of 1400 KPU/kg of curd solids is added to the first sample and second samples. The first sample is treated with the enzyme preparation for 1 hour, and the second sample is treated with the enzyme preparation for 2 hours. Following the enzyme treatment of the samples, the samples are thoroughly washed and the enzymes are thermally deactivated by jet cooking the samples at a temperature of 150° C. The samples are flash cooled to 50° C. by ejection into a vacuumized chamber. The cooled samples are then spray dried.

A control sample having a total solids content of 7.6% is provided from the initial protein slurry for comparison purposes. The control sample is heated to 50° C. for 1 hour, and then is thoroughly washed. The washed control sample is jet cooked at 150° C. and then is flash cooled in a vacuumized chamber to 52° C. The control sample is then spray dried.

The samples are analyzed to determine the ribonucleic acid content and the phytic acid content of the samples. The results of the analysis are shown in Table 3 below.

TABLE 3

| Enzyme treatment | | Ribonucleic acid | % reduction of |
|---|---|---|---|
| time | Phytic acid (%) | (mg/kg) | ribonucleic acid |
| t = 0 (control) | 1.41 | 9143 | — |
| t = 1 hour | 0.18 | 1769 | 80.7 |
| t = 2 hours | <0.06 | 1759 | 80.7 |

Treatment of soy protein isolate with an enzyme preparation containing an acid phosphatase and a phytase for a period of 1 hour or 2 hours is effective to substantially reduce the ribonucleic acid content and the phytic acid content in the protein isolate. Treatment for 2 hours increases the reduction of phytic acid content in the protein isolate relative to a 1 hour treatment, but does not significantly increase the reduction of ribonucleic acid content in the protein.

### EXAMPLE 6

The effect of temperature on the enzymatic degradation of ribonucleic acids and phytic acid in soy protein isolate by an enzyme preparation containing an acid phosphatase and a phytase enzyme is examined, where the soy protein isolate is purified in accordance with the present invention. A slurry is formed of soy protein isolate by mixing sufficient soy protein isolate with water adjusted to pH 4.5 by hydrochloric acid to form a slurry containing about 8% of the soy protein isolate by weight.

A first sample of the slurry containing 8% total solids by weight is prepared from the protein isolate slurry for enzymatic degradation of the ribonucleic acids and phytic acid. The first sample is adjusted to a temperature of 50° C. A second sample of the slurry containing 4% total solids by weight is prepared from the protein isolate slurry. The second sample is adjusted to a temperature of 38° C. These samples are then treated for two hours with an enzyme preparation containing an acid phosphatase enzyme and a phytase enzyme and having an activity of 1400 KPU/kg of curd solids. Following the enzyme treatment of the samples, the samples are thoroughly washed and the enzymes are thermally deactivated by jet cooking at a temperature of 150° C. The samples are flash cooled to 53° C. by ejection into a vacuumized chamber. The cooled samples are then spray dried.

A control sample having a total solids content of 7.6% is provided from the initial protein slurry for comparison purposes. The control sample is heated to 50° C. for 1 hour, and then is thoroughly washed. The washed control sample is jet cooked at 150° C. and then is flash cooled in a vacuumized chamber to 52° C. The control sample is then spray dried.

The samples are analyzed to determine the ribonucleic acid content and the phytic acid content of the samples. The results of the analysis are shown in Table 4 below.

TABLE 4

| | | Ribonucleic acid | % ribonucleic acid |
|---|---|---|---|
| Temperature | Phytic acid (%) | (mg/kg) | reduction |
| Control | 1.41 | 9143 | — |
| 50° C. | <0.07 | 1386 | 84.9 |
| 38° C. | 0.4 | 3848 | 58.0 |

Treatment of soy protein isolate with an enzyme preparation containing an acid phosphatase and a phytase at temperatures of 38° C. and 50° C. is effective to significantly reduce the ribonucleic acid content and the phytic acid content in the protein isolate. Treatment at 50° C. increases the reduction of phytic acid content and ribonucleic acid content in the protein isolate relative to treatment at 38° C.

### EXAMPLE 7

The effect of enzymatic degradation of phytic acid and ribonucleic acids in soy protein isolate by an enzyme preparation containing an acid phosphatase and a phytase on the mineral content in the protein is examined. In particular, the effect of the enzymatic degradation on the calcium, iron, magnesium, sodium, zinc, copper, potassium, manganese, and phosphorus concentrations in soy protein isolate is examined.

A slurry is formed of soy protein isolate by mixing sufficient soy protein isolate with water adjusted to pH 4.6 by hydrochloric acid to form a slurry containing about 8% of the soy protein isolate by weight. A sample is prepared for enzymatic degradation of phytic acid and ribonucleic acids from the slurry, where the sample weighs 3202 lbs. and has a total solids concentration of 7.6% by weight. The sample is heated to a temperature of 50° C. An enzyme preparation containing an acid phosphatase and a phytase and having an activity of 1400 KPU/(kg of curd solids) is added to the sample, and the sample is treated with the enzyme preparation for 1 hour. Following the enzyme treatment of the sample, the sample is thoroughly washed and the enzymes are thermally deactivated by jet cooking the sample at a temperature of 150° C. The sample is flash cooled to 53° C. by ejection into a vacuumized chamber. The cooled sample is then spray dried.

A control sample having a total solids content of 7.6% is provided from the initial protein slurry for comparison purposes. The control sample is heated to 50° C. for 1 hour, and then is thoroughly washed. The control sample is then jet cooked at 150° C. and then is flash cooled in a vacuumized chamber to 52° C. The control sample is then spray dried.

The samples are analyzed to determine the calcium, iron, magnesium, sodium, zinc, copper, potassium, manganese, and phosphorus content of the samples. The results of the analysis are shown in Table 5 below.

TABLE 5
ppm

| Sample | Ca | Fe | Mg | Na | Zn | Cu | K | Mn | P |
|---|---|---|---|---|---|---|---|---|---|
| Control | 1820 | 149 | 587 | 9070 | 33 | 12.4 | 8225 | 9.9 | 7991 |
| Sample | 1617 | 113 | 508 | 5988 | 26.8 | 11.7 | 5714 | 6.4 | 2583 |

Treatment of soy protein isolate with an enzyme preparation containing an acid phosphatase and a phytase is effective to reduce the calcium, iron, magnesium, sodium, zinc, copper, potassium, manganese, and phosphorus contents in the protein isolate. The enzymatic treatment is particularly effective in reducing the sodium, potassium, and phosphorus contents in the protein material.

It will be appreciated by those skilled in the art that various changes may be made in the invention as disclosed without departing from the spirit of the invention. The invention is not to be the specifics of the disclosed embodiments, which are for the purpose of illustration, but rather is to be limited only by the scope of the appended claims and their equivalents.

What is claimed:

1. A method for producing a purified vegetable protein material having low concentrations of ribonucleic acids, comprising:
forming an aqueous slurry of a vegetable protein material;
treating the slurry with an acid phosphatase enzyme at a temperature, a pH, and for a time period effective to degrade ribonucleic acids in the vegetable protein material; and
washing the treated slurry to provide a vegetable protein material having a reduced concentration of ribonucleic acids.

2. The method of claim 1 wherein said vegetable protein material is a vegetable protein concentrate or a vegetable protein isolate.

3. The method of claim 2 wherein said vegetable protein material is a soy protein concentrate or a soy protein isolate.

4. The method of claim 1 wherein said slurry contains from about 2% to about 30% of the protein material by weight.

5. The method of claim 4 wherein said slurry contains from about 5% to about 20% of the protein material by weight.

6. The method of claim 4 wherein said slurry contains from about 10% to about 18% of the protein material by weight.

7. The method of claim 1 wherein treatment of said slurry with said enzyme is effective to degrade a majority of ribonucleic acids in said vegetable protein material.

8. The method of claim 7 wherein washing the treated slurry is effective to remove said degraded ribonucleic acids to provide a vegetable protein material from which a majority of ribonucleic acids have been removed.

9. The method of claim 1 wherein treatment of said slurry with said enzyme is effective to degrade at least 60% of ribonucleic acids in said vegetable protein material.

10. The method of claim 9 wherein washing the treated slurry is effective to remove said degraded ribonucleic acids to provide a vegetable protein material from which at least 60% of ribonucleic acids have been removed.

11. The method of claim 1 wherein treatment of said slurry with said enzyme is effective to degrade at least 70% of ribonucleic acids in said vegetable protein material.

12. The method of claim 11 wherein washing the treated slurry is effective to remove said degraded ribonucleic acids to provide a vegetable protein material from which at least 70% of ribonucleic acids have been removed.

13. The method of claim 1 wherein treatment of said slurry with said enzyme is effective to degrade at least 80% of ribonucleic acids in said vegetable protein material.

14. The method of claim 13 wherein washing the treated slurry is effective to remove said degraded ribonucleic acids to provide a vegetable protein material from which at least 80% of ribonucleic acids have been removed.

15. The method of claim 1 wherein treatment of said slurry with said enzyme is effective to degrade substantially all of ribonucleic acids in said vegetable protein material.

16. The method of claim 15 wherein washing the treated slurry is effective to remove said degraded ribonucleic acids to provide a vegetable protein material from which substantially all of ribonucleic acids have been removed.

17. The method of claim 1 wherein treatment of said slurry with said enzyme is effective to degrade phytic acid and phytates in said vegetable protein material.

18. The method of claim 17 wherein washing the treated slurry is effective to remove said degraded phytic acid and phytates to provide a vegetable protein material from which phytic acid and phytates have been removed.

19. The method of claim 1 wherein said slurry is treated with an acid phosphatase at a pH of from about 3 to about 6.

20. The method of claim 19 wherein said slurry is treated with an acid phosphatase at a pH of from about 3.5 to about 5.5.

21. The method of claim 19 wherein said slurry is treated with an acid phosphatase at a pH of from about 4 to about 5.

22. The method of claim 19 wherein said slurry is treated with an acid phosphatase at a pH of from about 4.4 to about 4.6.

23. The method of claim 1 wherein said slurry is treated with an acid phosphatase at a temperature of from about 20° C. to about 70° C.

24. The method of claim 23 wherein said slurry is treated with an acid phosphatase at a temperature of from about 40° C. to about 55° C.

25. The method of claim 1 wherein said slurry is treated with an acid phosphatase wherein said acid phosphatase has an activity of about 600 KPU/g of curd solids to about 1400 KPU/g of curd solids.

26. The method of claim 1 wherein said slurry is treated with an acid phosphatase wherein said acid phosphatase is present in said slurry from about 0.1% to about 10% of the protein material by dry weight.

27. The method of claim 26 wherein said slurry is treated with an acid phosphatase wherein said acid phosphatase is present in said slurry from about 0.3% to about 5% of the protein material by dry weight.

28. The method of claim 1 wherein said slurry is treated with an acid phosphatase for a period of from about 30 minutes to about 4 hours.

29. The method of claim 28 wherein said slurry is treated with an acid phosphatase for a period of from about 45 minutes to about 3 hours.

30. The method of claim 1 further comprising a step of drying said treated and washed slurry to provide a purified vegetable protein material.

31. The method of claim 1 further comprising a step of heat treating said treated slurry.

32. The method of claim 1 further comprising a step of treating said washed and acid phosphatase treated vegetable protein slurry with a protease enzyme at a temperature, pH, and for a time sufficient to hydrolyze said protein in said slurry.

33. The method of claim 32 wherein said protease enzyme is present in said slurry in a concentration of from about 0.1% to about 10% of the protein in said slurry by dry weight.

34. The method of claim 32 further comprising the step of heat treating the hydrolyzed protein slurry.

35. The method of claim 32 further comprising the step of drying the hydrolyzed protein material after hydrolysis with said protease enzyme.

36. The method of claim 1 wherein said treatment of said vegetable protein material slurry with an acid phosphatase and said wash of said treated slurry are effective to lower the mineral content in the vegetable protein material.

37. A method for producing a purified vegetable protein material having low concentrations of ribonucleic acids, phytic acid, and phytates, comprising:
forming an aqueous slurry of a vegetable protein material;
treating the slurry with an acid phosphatase enzyme and a phytase enzyme at a temperature, a pH, and for a time period effective to degrade ribonucleic acids, phytic acid, and phytates in the vegetable protein material; and
washing the treated slurry to provide a vegetable protein material having reduced concentrations of ribonucleic acids, phytic acid, and phytates.

38. The method of claim 37 wherein said vegetable protein material is a vegetable protein concentrate or a vegetable protein isolate.

39. The method of claim 38 wherein said vegetable protein material is a soy protein concentrate or a soy protein isolate.

40. The method of claim 37 wherein said slurry contains from about 2% to about 30% of the protein material by weight.

41. The method of claim 40 wherein said slurry contains from about 5% to about 20% of the protein material by weight.

42. The method of claim 40 wherein said slurry contains from about 10% to about 18% of the protein material by weight.

43. The method of claim 37 wherein treatment of said slurry with said enzymes is effective to degrade a majority of ribonucleic acids in said vegetable protein material.

44. The method of claim 43 wherein washing the treated slurry is effective to remove said degraded ribonucleic acids to provide a vegetable protein material from which a majority of ribonucleic acids have been removed.

45. The method of claim 37 wherein treatment of said slurry with said enzymes is effective to degrade at least 60% of ribonucleic acids in said vegetable protein material.

46. The method of claim 45 wherein washing the treated slurry is effective to remove said degraded ribonucleic acids to provide a vegetable protein material from which at least 60% of ribonucleic acids have been removed.

47. The method of claim 37 wherein treatment of said slurry with said enzymes is effective to degrade at least 70% of ribonucleic acids in said vegetable protein material.

48. The method of claim 47 wherein washing the treated slurry is effective to remove said degraded ribonucleic acids to provide a vegetable protein material from which at least 70% of ribonucleic acids have been removed.

49. The method of claim 37 wherein treatment of said slurry with said enzymes is effective to degrade at least 80% of ribonucleic acids in said vegetable protein material.

50. The method of claim 49 wherein washing the treated slurry is effective to remove said degraded ribonucleic acids to provide a vegetable protein material from which at least 80% of ribonucleic acids have been removed.

51. The method of claim 37 wherein treatment of said slurry with said enzymes is effective to degrade substantially all of ribonucleic acids in said vegetable protein material.

52. The method of claim 51 wherein washing the treated slurry is effective to remove said degraded ribonucleic acids to provide a vegetable protein material from which substantially all of ribonucleic acids have been removed.

53. The method of claim 37 wherein treatment of said slurry with said enzymes is effective to degrade phytic acid and phytates in said vegetable protein material.

54. The method of claim 53 wherein washing the treated slurry is effective to remove said degraded phytic acid and phytates to provide a vegetable protein material from which phytic acid and phytates have been removed.

55. The method of claim 53 wherein treatment of said slurry with said enzymes is effective to degrade at least a majority of phytic acid and phytates in said vegetable protein material.

56. The method of claim 55 wherein washing the treated slurry is effective to remove said degraded phytic acid and phytates to provide a vegetable protein material from which at least a majority of phytic acid and phytates have been removed.

57. The method of claim 53 wherein treatment of said slurry with said enzymes is effective to degrade at least 75% of phytic acid and phytates in said vegetable protein material.

58. The method of claim 57 wherein washing the treated slurry is effective to remove said degraded phytic acid and phytates to provide a vegetable protein material from which at least 75% of phytic acid and phytates have been removed.

59. The method of claim 53 wherein treatment of said slurry with said enzymes is effective to degrade at least 85% of phytic acid and phytates in said vegetable protein material.

60. The method of claim 59 wherein washing the treated slurry is effective to remove said degraded phytic acid and phytates to provide a vegetable protein material from which at least 85% of phytic acid and phytates have been removed.

61. The method of claim 53 wherein treatment of said slurry with said enzymes is effective to degrade substantially all of phytic acid and phytates in said vegetable protein material.

62. The method of claim 61 wherein washing the treated slurry is effective to remove said degraded phytic acid and phytates to provide a vegetable protein material from which substantially all of phytic acid and phytates have been removed.

63. The method of claim 37 wherein said slurry is treated with an acid phosphatase and a phytase at a pH of from about 3 to about 6.

64. The method of claim 63 wherein said slurry is treated with an acid phosphatase and a phytase at a pH of from about 3.5 to about 5.5.

65. The method of claim 63 wherein said slurry is treated with an acid phosphatase and a phytase at a pH of from about 4 to about 5.

64. The method of claim 63 wherein said slurry is treated with an acid phosphatase and a phytase at a pH of from about 4.4 to about 4.6.

65. The method of claim 37 wherein said slurry is treated with an acid phosphatase and a phytase at a temperature of from about 20° C. to about 70° C.

66. The method of claim 65 wherein said slurry is treated with an acid phosphatase and a phytase at a temperature of from about 40° C. to about 55° C.

67. The method of claim 37 wherein said slurry is treated with an enzyme preparation containing an acid phosphatase and a phytase wherein said enzyme preparation has an activity of about 600 KPU/g of curd solids to about 1400 KPU/g of curd solids.

68. The method of claim 37 wherein said slurry is treated with an enzyme preparation containing an acid phosphatase and a phytase wherein said enzyme preparation is present in said slurry from about 0.1% to about 10% of the protein material by dry weight.

69. The method of claim 68 wherein said enzyme preparation is present in said slurry from about 0.3% to about 5% of the protein material by dry weight.

70. The method of claim 37 wherein said slurry is treated with an enzyme preparation containing an acid phosphatase and a phytase for a period of from about 30 minutes to about 4 hours.

71. The method of claim 70 wherein said slurry is treated with said enzyme preparation for a period of from about 45 minutes to about 3 hours.

72. The method of claim 37 further comprising a step of drying said treated and washed slurry to provide a purified vegetable protein material.

73. The method of claim 37 further comprising a step of heat treating said enzymatically treated slurry.

74. The method of claim 37 further comprising a step of treating said washed and enzymatically treated vegetable protein slurry with a protease enzyme at a temperature, pH, and for a time sufficient to hydrolyze said protein in said slurry.

75. The method of claim 74 wherein said protease enzyme is present in said slurry in a concentration of from about 0.1% to about 10% of the protein in said slurry by dry weight.

76. The method of claim 74 further comprising the step of heat treating the hydrolyzed protein slurry.

77. The method of claim 74 further comprising the step of drying the hydrolyzed protein material after hydrolysis with said protease enzyme.

78. The method of claim 37 wherein said treatment of said vegetable protein material slurry with an acid phosphatase and a phytase and said wash of said treated slurry are effective to lower the mineral content in the vegetable protein material.
